(19) United States
(12) Patent Application Publication
(10) Pub. No.: US 2004/0076559 A1
(43) Pub. Date: Apr. 22, 2004

(54) Device and method for the production of singlet oxygen

(76) Inventors: Franz Brucker, Freiburg (DE); Michael Koehl, Britzingen (DE); Volker Lieske, Freiburg (DE)

Correspondence Address: Mark A. Hixon, Marshall & Melhorn, LLC, 8th Floor, Four Seagate, Toledo, OH, 43604, US

(21) Appl. No.: 10/638,880

(22) Filed: Aug. 11, 2003

Related U.S. Application Data
(63) Continuation-in-part of application No. 09/762,158, filed on Mar. 28, 2001, now abandoned.

(30) Foreign Application Priority Data
Aug. 5, 1999 (WO) PCT/EP99/05667
Aug. 5, 1998 (DE) 198 35 456.8

(51) Int. Cl. B01J 19/08

(52) U.S. Cl. 422/186; 422/186.07; 204/157.15; 204/157.3

(57) ABSTRACT

A device for producing singlet oxygen and having a light source and a transparent medium is described. The transparent medium has a first surface and second surface, the first surface facing the light source and a region which contains a photosensitizer are disposed on the second surface. The transparent medium is completely closed against a surrounding medium.

### CROSS-REFERENCE TO RELATED APPLICATIONS

This application is a continuation-in-part application of U.S. patent application Ser. No. 09/762,158, which was filed on Mar. 28, 2001. U.S. patent application Ser. No. 09/762,158 was pending as of the filing date of the present application. U.S. application Ser. No. 09/762,158 is hereby incorporated by reference as if set forth in its entirety herein.

### BACKGROUND OF THE INVENTION

The invention relates to a device and a method for producing singlet oxygen from triplet oxygen.

In its basic state oxygen exists as a triplet molecule and can be transferred into the excited singlet state by having energy supplied to it. Excited singlet oxygen is particularly highly reactive and is used for example in chemical process engineering, in medicine and in water treatment.

Up to now singlet oxygen has been produced either through chemical reactions or through irradiation of a photosensitive layer which is located in contact with air or pure oxygen. Through quenching the fluorescence, the triplet oxygen molecules are transformed by radiationless transition into the excited singlet state. Since singlet oxygen only has a very short lifespan in solution it is generally brought to the place of use in a gaseous state.

In cancer therapy it is usual to distribute the photosensitizer in the body through an intravenous injection. Then, to produce singlet oxygen locally, light is coupled by endoscopy in the region of the diseased tissue. With skin diseases, following an injection of the photosensitizer, the diseased part of the skin is irradiated from the outside with light in order to produce locally singlet oxygen. Injecting the photosensitizer has the disadvantage, however, that the patient, as a result of side effects as a consequence of uncontrolled irradiation, has to spend at least four weeks in darkened rooms following the treatment, until the photosensitizer has been broken down or excreted.

### OBJECT OF THE INVENTION

Proceeding from this problem, the object underlying the invention is to quote a device for producing singlet oxygen which permits local production of singlet oxygen which can be metered better. The device should in particular permit the singlet oxygen treatment of patients with fewer side effects. The object of the invention is furthermore to quote a method for the local production of a singlet oxygen.

These objects are accomplished by a device for producing singlet oxygen comprising a light source and a transparent medium which has a first surface and a second surface. It is possible for light from the light source to be coupled into the transparent medium via the first surface. A region which contains at least one photosensitizer is disposed on the second surface. The transparent medium is completely closed against a surrounding medium.

Additional embodiments are accomplished by a method of producing singlet oxygen from triplet oxygen, comprising the steps of:

a) generating light;

b) coupling the light into a transparent medium;

c) guiding the light within the transparent medium to a surface of the transparent medium, on which a region is disposed containing at least one photosensitizer and which is located in contact with triplet oxygen and is in direct contact with the surrounding medium; and

d) photoinduced excitation of the triplet oxygen to form singlet oxygen with the aid of the photosensitizer.

Other preferred embodiments of the present invention are disclosed herein.

### SUMMARY OF THE INVENTION

A device for producing singlet oxygen from triplet oxygen is proposed which contains a light source and a transparent medium which has a first surface and a second surface. Whilst light from the light source is coupled via the first service of the transparent medium, the second surface is provided with a region containing one or more photosensitizers. This region can be a coating or a carrier for the photosensitizer which is disposed on the second surface and contains the photosensitizer, e.g. in the form of a coating, or embedded in a possibly porous matrix. In order to increase its surface area, the region can also have a roughened surface.

To produce singlet oxygen, the generated light is first coupled via the first surface into the transparent medium and is led within the transparent medium to the second surface. In the vicinity of the second medium, the light guided in the transparent medium comes into contact with the photosensitizer which in turn is located in contact with triplet oxygen. As a result of an interaction between photosensitizer, triplet oxygen and light, singlet oxygen is formed locally in the region of the second surface of the transparent medium.

The local production, according to the invention, of singlet oxygen in the region of the second surface of the transparent medium is connected with a number of advantages. Thus, for example, when the device according to the invention is used in the medical field, an intravenous injection of the photosensitizer can be dispensed with. Apart from in the medical field, the invention can however also be applied to many other fields which require the local production of singlet oxygen. Thus for example its use in chemical process engineering can be thought of.

For large-area production of singlet oxygen, the transparent medium can for example be configured as a transparent body in the form of a covering surrounding the light source at least in regions. Alternatively, the transparent medium can be an integral component of the light source such as for example the bulb of a lamp or some other covering securely connected to the lamp. The transparent medium is brought with the surface on which the photosensitizer is arranged into the vicinity of the place of use and is illuminated on the other surface by the light source.

For the local production of singlet oxygen in regions that are difficult to access, the transparent medium can be configured as a light guide which is provided on an end face with a region containing the photosensitizer, it being possible for the light to be coupled by endoscopy via the other end face.

### BRIEF DESCRIPTION OF THE DRAWINGS

Further details and preferred developments of the invention arise from the figures and the embodiments. The figures show:

FIG. 1 a device for the local large-area production of singlet oxygen;

FIG. 2 a device for the local production of singlet oxygen in regions which are difficult to access; and

FIG. 3 a device for the local production of singlet oxygen with a detachable carrier for the photosensitizer.

### DESCRIPTION OF THE PREFERRED EMBODIMENTS

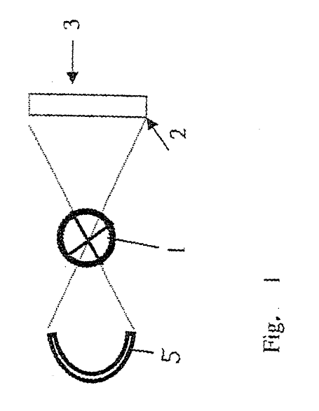

In FIG. 1 is represented a device for the large-area local production of singlet oxygen having a light source 1, a planar transparent body 2 and a layer 3 containing a photosensitizer. As the light source 1 can be used a wide-band lamp, e.g. in conjunction with a reflector 5, a laser, e.g. in conjunction with a diffuser, or even the sun. The transparent body 2, which is provided with a photosensitizer layer 3 on its front side, is irradiated from the rear side with light from the light source 1. The device according to FIG. 1 is suitable e.g. for treating skin diseases.

Instead of applying the layer 3 directly to a surface of the transparent body 2, consideration can also be given to applying the layer 3 to an e.g. transparent carrier, which is disposed detachably on the surface of the transparent body 2.

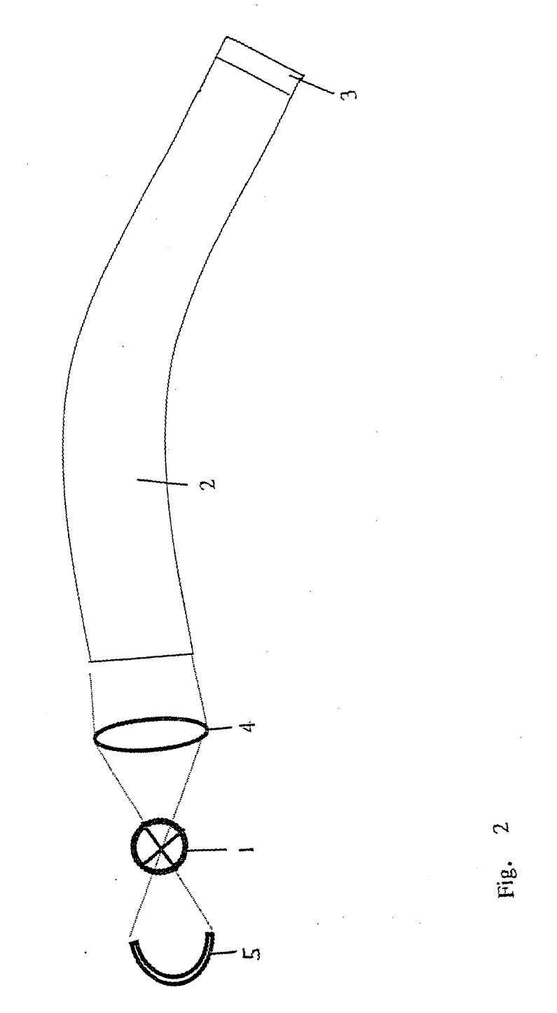

In FIG. 2 is represented a device for the local production of the singlet oxygen in regions which are difficult to access, e.g. inside the body. The device contains a light source 1 and a glass fibre as a light guide 2. With the aid of a coupling optical system 4, e.g. a lens, the light generated by the light source 1 is coupled via an end face of the light guide into the light guide. On the other end face of the light guide 2 is applied a layer 3 containing a photosensitizer or a carrier which is exchangeable and contains a photosensitizer. This end face is then guided to the place of application for treatment.

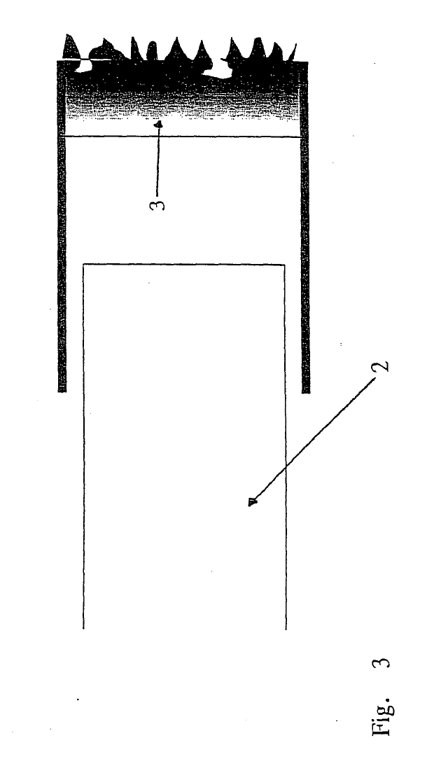

In FIG. 3 is depicted a modification of the device represented in FIG. 2. The region containing the photosensitizer is configured as the carrier 3 which is placed on the light guide 2. The carrier 3 includes a mounting tube, which is closed at one end face with a substrate containing the photosensitizer. The substrate surface is roughened in order to increase the active surface for the production of singlet oxygen. Basically the carrier 3 can contain e.g. glass substrates, porous glass filters or transparent polymers which are e.g. coated on the outer side with the photosensitizer or are mixed in their entire volume with the photosensitizer. The carrier can be connected mechanically or in an adhesive manner to the transparent medium 2.

The layer 3 shown in FIGS. 1, 2 and 3, or the carrier 3, contains as the photosensitizer e.g. a substance which is selected from Rose Bengal, copper(II)-phthalocyanine, 5-aminolevulinic acid, porphyrins, phthalocyanines, chlorines, tetraphenylporphyrins, benzoporphyrin derivatives, purpurins, pheophorbides and metal complexes of same. For the use of the devices in an environment which is low in oxygen, the region 9 can be configured porous for storing oxygen. Thus, for example, a porous photosensitizer or even a porous carrier for the photosensitizer can be used. In addition, together with the photosensitizer, an oxygen-donating chemical compound such as for example organic or inorganic peroxides can be applied to the transparent medium 2. Oxygen can also be supplied to layer 3 through free convection or from storage containers. In the device shown in FIG. 2, a hose guided with the light guide can make possible the direct supply of oxygen to the photosensitizer.

Instead of oxygen, in the devices according to FIGS. 1 to 3, an oxygenous gas, such as in particular air, can be supplied to layer 3. Photosensitizer can also be supplied subsequently.

In a preferred embodiment of the present invention, a device for producing singlet oxygen comprises a light source and a transparent medium which has a first surface and a second surface. It is possible for light from the light source to be coupled into the transparent medium via the first surface. A region which contains at least one photosensitizer is disposed on the second surface. The transparent medium is completely closed against a surrounding medium. In other words, the transparent medium can form a closed system, which is separated from the surrounding atmosphere, or any other surrounding medium.

A preferred embodiment of practicing the method of the present invention, includes the steps of:

a) generating light;

b) coupling the light into a transparent medium;

c) guiding the light within the transparent medium to a surface of the transparent medium, on which a region is disposed containing at least one photosensitizer and which is located in contact with triplet oxygen and is in direct contact with the surrounding medium, wherein the transparent medium is completely closed against the surrounding medium; and

d) photoinduced excitation of the triplet oxygen to form singlet oxygen with the aid of the photosensitizer.

In a preferred embodiment of the present invention, the source of natural light for use in the invention is sunlight, or possibly moonlight.

1. A device for producing singlet oxygen comprising a light source and a transparent medium which has a first surface and a second surface, it being possible for light from the light source to be coupled into the transparent medium via the first surface, and a region which contains at least one photosensitizer being disposed on the second surface, wherein the transparent medium is completely closed against a surrounding medium.

2. A device according to claim 1, further comprising a device for supplying oxygen and/or an oxygenous gas and/or photosensitizer to the region.

3. A device according to claim 1, wherein the light source is natural light, a lamp or a laser.

4. A device according to claim 1, wherein the photosensitizer is selected from porphyrins, phthalocyanines, chlorines, tetraphenylporphyrins, benzoporphyrin derivatives, purpurins, pheophorbides and metal complexes of same.

5. A device according to claim 1, wherein the photosensitizer is copper(II)-phthalocyanine, Rose Bengal or 5-aminolevulinic acid.

6. A device according to claim 1, wherein the region (3) contains an oxygen-donating chemical compound.

7. A device according to claim 1, wherein the region (3) is porous and/or has a rough surface.

8. A device according to claim 1, wherein the region (3) containing the photosensitizer is configured as a coating or as a carrier.

9. A device according to claim 1, wherein the transparent medium (2) is an integral component of the light source (1).

10. A device according to claim 1, wherein the transparent medium (2) is a covering surrounding the light source (1) at least in regions.

11. A device according to claim 1, wherein the transparent medium (2) is a light guide and the first surface is formed by a first end face of the light guide, and the second surface is formed by a second end face of the light guide.

12. A device according to claim 11, wherein a coupling lens system (4) is disposed in front of the first surface to couple the light of the light source (1).

13. A device according to claim 11, wherein a device which can be guided together with the light guide is provided to emit oxygen and/or an oxygenous gas and/or photosensitizer.

14. A method of producing singlet oxygen from triplet oxygen, comprising the steps of:
a) generating light;
b) coupling the light into a transparent medium (2);
c) guiding the light within the transparent medium (2) to a surface of the transparent medium (2), on which a region (3) is disposed containing at least one photosensitizer and which is located in contact with triplet oxygen and is in direct contact with the surrounding medium; and
d) photoinduced excitation of the triplet oxygen to form singlet oxygen with the aid of the photosensitizer.

15. A method according to claim 14, wherein oxygen and/or an oxygenous gas and/or photosensitizer is guided to region (3).

16. A method according to claim 14 wherein the transparent medium (2) is used as a covering, an integral component of a light source (1) or a light guide.

17. A method according to claim 14, wherein the light is coupled into the transparent medium with the aid of a coupling lens system (4).

18. A method according to claim 14, wherein oxygen and/or an oxygenous gas is stored in the region (3) containing the photosensitizer.

19. A method according to claim 14, wherein region (3) is configured porous and/or provided with a porous surface.

20. A device according to claim 6, wherein the an oxygen-donating chemical compound comprises organic or inorganic peroxides.
